(19) United States
(12) Patent Application Publication
(10) Pub. No.: US 2024/0377309 A1
(43) Pub. Date: Nov. 14, 2024

(54) Background hotspots reduction for plasmon-enhanced fluorescence biosensing

(71) Applicants: Canon Medical Systems Corporation, Tochigi (JP); The General Hospital Corporation, Boston, MA (US)

(72) Inventors: Satoru Komatsu, Melrose, MA (US); Hyungsoon Im, Weston, MA (US)

(21) Appl. No.: 18/661,090

(22) Filed: May 10, 2024

Related U.S. Application Data
(60) Provisional application No. 63/501,874, filed on May 12, 2023.

(51) Int. Cl. G01N 15/1433; G01N 33/543; G01N 33/569

(52) U.S. Cl. CPC G01N 15/1433; G01N 33/54373; G01N 33/569

(57) ABSTRACT

An optical imaging system and method are provided for use with plasmon-enhanced optical imaging. The imaging system includes a multi-channel fluorescent microscope configured to image a sample on a plasmonic substrate; an image acquisition control unit configured to acquire multiple images at different times in a first channel; and an image processing unit configured to distinguish a signal within one or more of the multiple images as either (1) a signal from the sample or (2) a signal from a background hotspot.

### CROSS-REFERENCE TO RELATED APPLICATION(S)

This application relates, and claims priority, to U.S. Patent Application Ser. No. 63/501,874 filed 12 May 2023, the disclosure of which is incorporated by reference herein in its entirety.

### FIELD OF THE DISCLOSURE

The present disclosure relates to plasmon enhanced biosensing. More particularly, the disclosure exemplifies plasmon-enhanced fluorescence biosensing where the images are processed to remove background hotspot noise.

### BACKGROUND INFORMATION

Extracellular vesicles (EVs) in bodily fluids have been considered promising materials for liquid biopsy by virtue of the EVs containing many kinds of proteins, miRNAs, and mRNAs on its surface or inside the vesicle. EVs are classified into several subtypes, which include exosomes, microvesicles, microparticles, ectosomes, oncosomes, apoptotic bodies, and so on, and their size has large variety with a range of around 50 nm to 5 μm. Liquid biopsy on small EVs below 200 nm have been actively studied, but most of the research uses bulk assay for analyzing EVs properties because EVs cannot be observed with an ordinary optical microscope due its small size. Fluorescence microscopy is one way to make single EVs observable, but for small EVs, the fluorescence intensity is weak and observation becomes difficult. One method to detect small single EVs is plasmon-enhanced fluorescence microscopy which uses a plasmon chip instead of a classical glass plate. Many kinds of plasmon chips have been developed, and some kinds of plasmon chips can cause unstable background hotspots which size and intensity are similar to signal from EVs.

Sensing using surface plasmons have been discussed, for example, by S. Park, et al., (“Self-Assembly of Nanoparticle-Spiked Pillar Arrays for Plasmonic Biosensing,” Adv. Funct. Mater., 2019, Vol. 29, Issue 43, 1904257); Keiko Tawa, et al., (“Optical microscopic observation of fluorescence enhanced by grating-coupled surface plasmon resonance,” Opt. Express 16, 9781-9790 (2008)); Taylor, AB and Zijlstra, P, (ACS Sens. 2017, 2, 8, 1103-1122); and U.S. Pat. No. 8,039,267. Surface plasmons are also described in WO 2021/211756, which is hereby incorporated by reference in its entirety.

### SUMMARY OF EXEMPLARY EMBODIMENTS

According to at least one embodiment of the disclosure, there is provided a system, apparatus, and methods that include an optical imaging system for plasmon-enhanced optical imaging comprising: a fluorescent microscope configured to image a sample on a plasmon substrate; an image acquisition control unit configured to acquire multiple images at different times in a first channel; and an image processing unit configured to distinguish a signal within one or more of the multiple images as either (1) a signal from the sample or (2) a signal from a background hotspot. This change may be based on a change over time in the one or more of the multiple images. The image processing unit distinguishing the signal by: executing a particle detection analysis on the first image and creating a base particle map, executing a particle detection analysis on the at least a second image(s) and creating a temporal particle map, defining a particle in the base particle map as a background hotspot when the particle has no correspond particle in the temporal particle map at a location within a location threshold of the particle, and defining a particle in the base particle map as a signal from the sample when the particle has a correspond particle in the temporal particle map at a location within a location threshold of the particle. The image processing unit may perform a number of these processes in an iterative manner so as to update the base particle map with information from additional images. The image processing unit may display, for example, the base particle map, or an updated particle map.

There is provided in yet other embodiments of the disclosure, a system, apparatus, and methods that includes an optical imaging system for plasmon-enhanced optical imaging comprising: a fluorescent microscope configured to image a sample on a plasmon substrate; an image acquisition control unit configured to acquire multiple images at different times in a first channel; and an image processing unit configured to distinguish a signal within one or more of the multiple images as either (1) a signal from the sample or (2) a signal from a background hotspot. This change may be based on a change over time in the one or more of the multiple images. The image acquisition control unit may be configured to set an acquisition time for the multiple images, where the acquisition time is set to be longer than a bleaching time of a florescent dye in the sample. In particular, the acquisition time may be between 100 ms and 5 s, or between 600 ms and 2 s, and there are at least 2, 4, 6, 8, 10 or more images acquired at different times in the first channel.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

Further objects, features and advantages of the present disclosure will become apparent from the following detailed description when taken in conjunction with the accompanying figures showing illustrative embodiments of the present disclosure.

FIG. 1 is a chart of the percentage considered noise for different exposure times.

FIG. 2 is a system diagram.

FIG. 3 provides a flowchart of one embodiment of the process.

FIG. 4 provides a flowchart for the background hotspot noise reduction.

FIGS. 5(A)-5(D) are examples of various particle maps.

FIG. 6 provides a flowchart showing one embodiment of the background hotspot noise reduction.

FIG. 7 provides a flowchart showing one embodiment of the background hotspot noise reduction.

FIG. 8 provides a flowchart showing one embodiment of the background hotspot noise reduction.

FIG. 9 provides a graph showing counts of detected background hotspot noise.

FIG. 10 provides a graph showing co-localization rates based on previous frame data and based on first frame data.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENTS

When applying the plasmon chip for sensitive virus detection using fluorescent dyes, tiny background hotspot noise does not significantly affect its analysis, because it the analysis is not single molecular analysis, but instead bulk detection. In a case of single EVs analysis, it is important to detect tiny signals separating each EV and the difficulty is that the size and intensity of these small signals are similar to the background hotspot noise that are occurring on the plasmon chip. Also, if the background hotspot noise is stable in time, it can be easily identified by measuring the position of occurrence in advance, however, the intensity is still modulated such as noise that is generated and disappears, and the generated position is random. When long exposures are used to improve signal to noise ratio (“SNR”), the blinking background is captured as a bright spot, indistinguishable from the signal from the EVs. Since bright spots are created while acquiring an image in locations where they do not occur initially, the pre-obtained background hotspots positions are not applicable for these reductions.

Thus, there is provided herein a system, apparatus, and method to reduce unnecessary background hotspot noise from a plasmon chip for fluorescent microscopy for detecting fluorescent signals from small amounts of fluorescent dyes on small biological samples such as extracellular vesicles.

### EVs Detection Using a Microscope

EVs cannot be resolved with an optical microscope due to their small size. In order to detect them with a microscope, a fluorescent dye is attached and its fluorescent light is detected as a bright spot to detect the presence or absence of the fluorescence signal. Because of the small size, there are few fluorescent dyes to attach to the EV, and the fluorescent signal is weak, so amplification of the signal is required. One method is to use plasmon resonance.

### Background Hotspot Noise

Some kinds of plasmon chips which use local plasmonic resonance effect have background hotspot noise which is a tiny bright signal similar to a signal from a sample. In addition, the location of the noise is random, with strong and weak signals, blinking irregularly or observed only once at a particular position. A pixel set larger than a minimum size, e.g., 3×3 or 2×2 pixels, specified by the pixel whose intensity exceeds the intensity threshold value is detected as the background hotspot noise. An exemplary plasmon chip made of gold shows a few hotspot noises from the wavelength band of the AF555 and numerous hotspot noises in the wavelength bands of the AF647 and Cy7. In more detail, when we observe the chip at the AF647 wavelength band, about 5% of the total number of noise will (in some embodiments such as 5 s of continuous shooting with an exposure time of 100 ms per frame) keep appearing in all frames and about 16% of them containing the above described non disappearing noise will appear only one time. Half of the noises blink 4 times or less during the 50 frames images, and 90% blink 10 times or less. The expected value of the numbers of noise blinking is approximately 5 under the above condition. Also, the expected maximum emission time for noise, excluding noise that always appears, is about 900 ms.

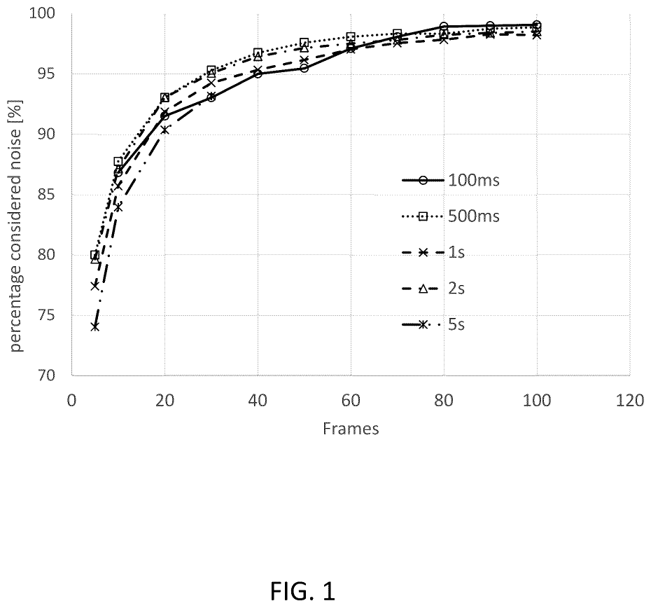

Shorter exposure time per frame results in less background hotspot noise detected but a lower SNR. On the other hand, a longer exposure time improves SNR but increases the detected noise. Therefore, it was examined how the detected background hotspot noise changed by changing the exposure time and the number of frames. The percentage of hotspots that blinked at least once during the entire frame time was examined and the results are shown in the FIG. 1. Here, exposure times were set to 100 ms, 500 ms, 1 s, 2 s, and 5 s. Longer exposure times increased the noise detected, but the percentage identified as hotspot noise was in the range of plus or minus 2% for the same number of frames. If noise is to be completely eliminated, several hundred frames are required to be acquired. The overall acquisition time, residual noise, and SNR should be taken into account when determining the exposure time and number of frames. Short exposure time is suitable for reducing the absolute number of the hotspot noise, but it must be balanced against the deterioration of SNR. Low SNR causes detection loss of weak signals from EVs. Allowable percentage of residual noise after a noise reduction method is one way to determine preferred the acquisition condition; it is possible to determine the optimal number of frames from FIG. 1. In other words, the required number of frames can be determined by determining the exposure time of one frame based on the SNR and the acceptable residual hotspot noise percentage.

Due to these characteristics, it is not possible to locate most of the noise in advance. However, if multiple images are captured, it is possible to distinguish noise from blinking bright spots and signal intensity decay.

### System Overview

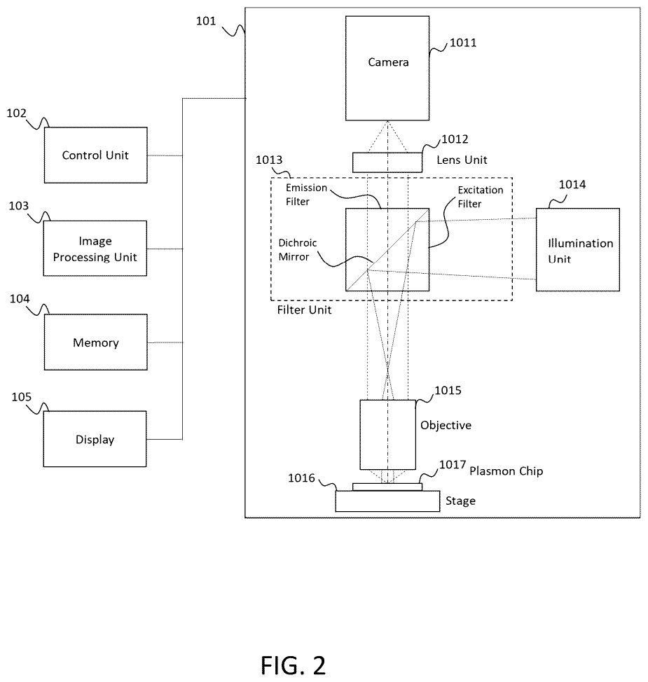

The imaging system as shown in FIG. 2 is an example of an imaging system having a fluorescent imaging system as described herein. The imaging system of FIG. 2 includes a fluorescent microscope 101, a control unit 102, an image processing unit 103, a memory unit 104, and a display unit 105. The fluorescent microscope comprises a camera 1011, a lens unit 1012, a filter unit 1013, an illumination unit 1014, an objective 1015, and a stage 1016. A sample on a plasmon chip 1017 is placed on the stage. The control unit 102 controls total system process which are acquiring images with controlling the fluorescent microscope, image processing, displaying images and storing data. The image processing unit 103 executes the background hotspot noise reduction using multiple images and outputs detected sample positions and/or identified the background hotspot noise positions. The memory unit 104 stores the acquired images temporarily and the calculated data. The display unit 105 shows data of the system such as the information of the microscope control, the live images, the acquired images, and the calculation results.

### Generalized Process

The intensity characteristics of noise are more variable than the intensity characteristics due to fluorescence, dropping below the detection level faster than the time it takes for fluorescence to bleach. On the other hand, unlike the bleaching of fluorescence, noise may drop below the detection level and then rise above the detection level again. This difference in intensity change characteristics is used to distinguish whether the detected bright spots are fluorescent signals or bright spot noise. For this purpose, multiple frames of images are acquired within a time period in which fluorescence does not bleach, and if the intensity of a bright spot is changed by more than a certain intensity threshold, the bright spot is identified as noise.

The plasmon chip as described herein may be any plasmonic surface that effects to enhance surface plasmon signals. An example of the plasmon chip is those described by Park et al., (“Self-Assembly of Nanoparticle-Spiked Pillar Arrays for Plasmonic Biosensing,” Adv. Funct. Mater., 2019, Vol. 29, Issue 43, 1904257) or in WO 2021/211756 herein incorporated by reference in its entirety. These plasmon chips are nano-plasmonic arrays configured to amplify one or more specific wavelengths of electromagnetic radiation. These plasmon chips may be gold nanorods imprinted on a resist layer and coated with a surface layer of citrate, PEG, etc. Other exemplary plasmon chips may be formed from, for example, silver or gold island films, gold nanospheres, gold bipyramids, etc.

### Use Particle Detection Result at Each Frame

To identify the background hotspot noise, the normal exposure time is split into shorter exposure times and multiple images are acquired to distinguish between the unstable noise signals and the more stable signals from samples.

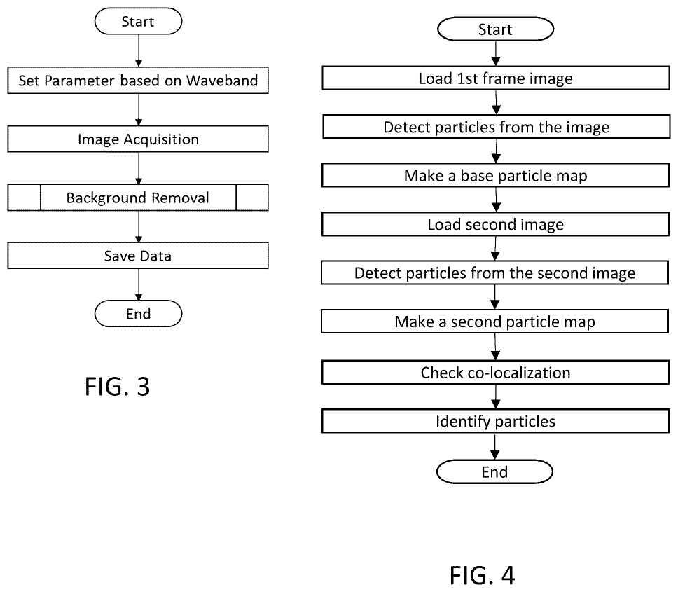

The process for particle detection in a single channel is explained by following the exemplary flowchart shown in FIG. 3.

First, a waveband of the fluorescent microscope is selected based on the wavelength that excites the fluorescent dye, and the shooting parameter (for image acquisition) is set depending on the waveband. The bleaching time of a fluorescent dye is different depending on the fluorescent dye and the illumination power; therefore total exposure time (a summation of each frames' exposure time), and illumination power are optimized based on the fluorescent dye.

There are several approaches to determining the exposure time and number of frames. One way to do that is a method using the results of above background noise analysis, where exposure time is set 900 ms which is the expected maximum noise duration time, and the number of frames is set 5 which is the expected number of relight of noise. Another way is a method based on the signal level of EVs. A short exposure time causes detection loss for weak signals from EVs. The exposure time should be set to the optimal exposure time that allows noise to be isolated while minimizing undetected EVs. To determine the optimal exposure time, images with different exposure times need to be acquired using EVs sample. The number of detected EVs and background hotspots will be increased according to the exposure time but the number of detected EVs will be saturated after a certain exposure time. Thus, the slope of the graph plotting the number of detection changes, and the exposure time at which the change occurs, can be regarded as the time at which all EVs can be detected. Here, the background noise is assumed to occur at random locations and to increase monotonically with exposure time. For example, the exposure time was around 600 ms at wavelengths between AF488 and Cy7 channels under the experiment described herein. In this case, the SNR is not so high, and some EV signals are missing; considering the SNR and the overall acquisition time, an exposure time of 1 to 2 seconds per frame is optimal under these experimental conditions. If the percentage of residual noise is set to 5%, it is preferable to acquire around 40 frames. Similarly, setting it to 10% or 2% would require around 15 or 80 frames respectively. Here, the absolute number of residual noises depends on the exposure time, so it is preferable to set it to 1 second.

Furthermore, in some embodiments, it is preferable to acquire images while changing wavelengths rather than continuously acquiring images at the same wavelength, as the possibility of noise generation position changes.

The total exposure time under certain illumination conditions must be shorter than the bleaching time of the fluorescent dye, because if it were bleached, it would be indistinguishable from the background hotspot noise. The bleaching time of individual fluorescent molecules is slightly different, and the bleaching time has a distribution even if the same fluorescent dye is used. The total exposure time must be short enough to avoid the impact of misdetection due to differences in the bleaching time. For example, the total exposure time should be shorter than the average of the bleaching time minus three standard deviations of the distribution of the bleaching time. Once the total exposure time is decided, the number of acquired images is decided based on the total exposure time and the exposure time of each frame.

Regarding the number of frames, the more frames used, the better the noise reduction, so it is better to use the maximum number of frames that will not bleach the phosphors to be attached to the EV. However, it can be reduced in consideration of measurement time and effectiveness. For example, if 5% remaining noise is acceptable, the total exposure time should be 5s based on the above example.

Next, image acquisition is executed based on the parameters which were decided in the previous step. Here, multiple image acquisitions may be performed for each wavelength in turn or alternately by changing wavelengths.

### Basic Workflow

After acquiring the multiple temporal images within the same fluorescence channel, the background noise reduction process is executed. A flowchart of a background noise reduction is shown in FIG. 4.

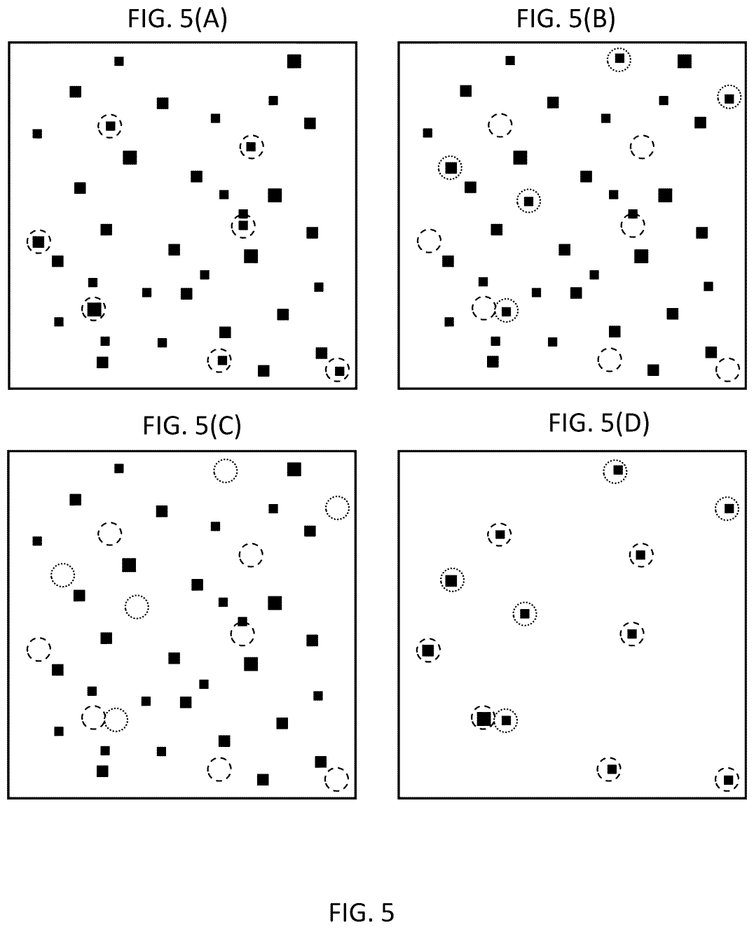

The image of the first frame is loaded from the memory unit and processed for particle detection which detects bright spots in the image as particles. Here, the first frame image contains not only signals from samples but also signals from the background hotspot noise. From the detection result, the base particle map with 0 and 1 values is generated and stored in memory. FIG. 5(a) shows an example of the base particle map, and the dot circles are the background hotspot noise. For more information, the map is generated with the center of gravity pixel of each detected particle as the center, with the rectangular area of the predefined minimum detection size of particles as 1 and the non-particle area as 0. Alternatively, the rectangular area bounded or inscribed by the detected particles is generated as 1 and the non-particle area is generated as 0.

Then, the second frame image is loaded, and a second particle map which also has 0 and 1 values is generated by applying the particle detection to the second image like same as the first image. FIG. 5(b) is an example of the second particle map, and the dot circles mean disappeared particle areas and the fine dot circles mean newly detected particle areas. The base particle map and the second particle map are compared to check whether particles are detected at same positions, which is called co-localization check, and particles in the base particle map that are present in the same position in the second particle map are identified as particles (e.g. EV particles) and are signal from the sample as opposed to hotspot noise. Whether particles are at the same position depends on when a particle in the base particle map has a correspond particle in the temporal particle map at a location within a location threshold of the particle. Particles that are in one of the base particle map and the temporal particle map but where a corresponding particle is not found in the other particle map within a location threshold of the particle are identified as hotspot noise.

For every particle in the base particle map, this process is performed to identify the particles not considered the background hotspot noise. FIG. 5(c) is an example of the updated base particle map and FIG. 5(d) is an example of a noise map, a map of particles identified as noise. Here, if each particle area in the two maps overlaps more than a set location threshold, it is determined that the particle exists at the same location between the two images. The location threshold should be more than half of the regions overlap. In some embodiments, the location threshold is more than 50%, 60%, or 70% of the regions overlap. The size of the detected particles may differ slightly, in which case the smaller particle is used as the basis for judgment.

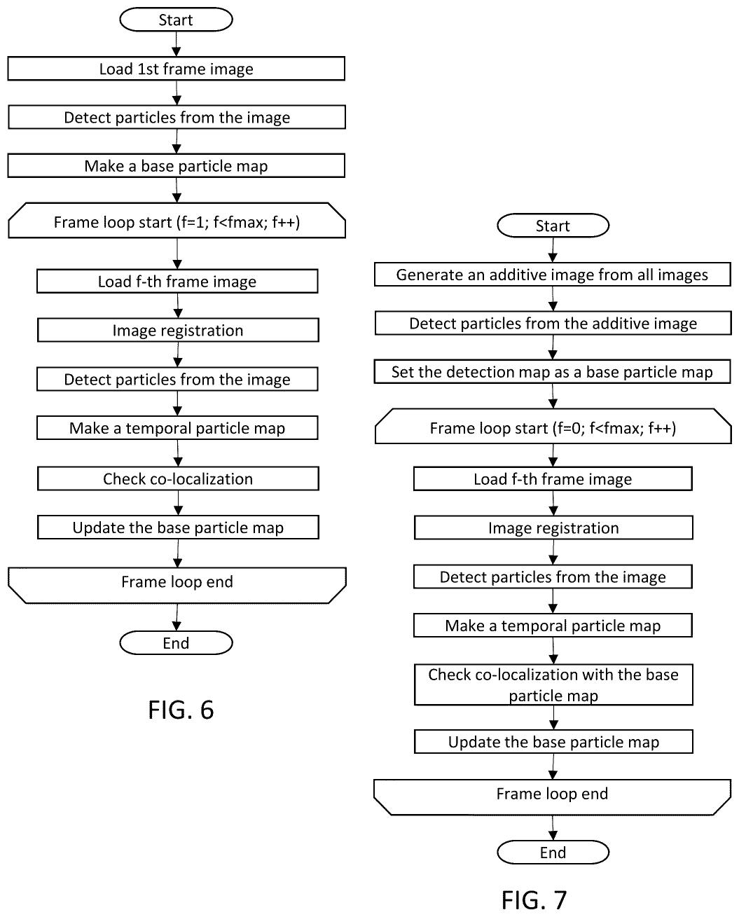

After acquiring the multiple images, the background noise reduction process is executed. A flowchart of the background noise reduction is shown in FIG. 6, in which an looped process is performed.

As before, the image of the first frame is loaded from the memory unit and processed for particle detection and a base particle map is created.

Then, the next frame image is loaded, and image registration between the first frame image and the current frame image is processed. This process can be omitted when the stage of the microscope is stable while acquiring the series of images. A temporal particle map is generated by applying the particle detection to the current image like same as the first image. The base particle map and the temporal particle map are compared to check whether particles are detected at same positions, which is called co-localization check, and particles in the base particle map that are not present in the same position in the temporal particle map are excluded from the base particle map. For every particle in the base particle map, this process is performed to produce the updated base particle map. FIG. 5(c) is an example of the updated base particle map and FIG. 5(d) is an example of a noise map, a map of particles identified as noise. This update process is continued with additional images until the final frame image. Here, if each particle area in the two maps overlaps more than a set location threshold, it is determined that the particle exists at the same location between frames. In some embodiments, the location threshold should be more than half of the regions overlap. In some embodiments, the location threshold is more than 50%, 60%, or 70% of the regions overlap. The size of the detected particles may differ slightly, in which case the smaller particle is used as the basis for judgment. If the co-localization check is performed, the process moves forward with the updated base particle map.

The updated base particle map is saved to the memory unit as the particle detection result. In addition, size information of particles, intensities of the detected particles, and some statistical information can be stored as useful data. Also, the acquired images are saved if these are necessary, but saving an image summed all frames or averaged all frames is better to reduce memory usage.

After saving the calculation results such as the updated base particle map, the process is completed. In this embodiment, the base particle map is generated from the first image, however, the summation image or the averaged image from all frames can be used for creating the base particle map. See FIG. 7. In this case, an image with better signal to noise ratio would be applied to create the base particle map.

### Use Particles' Intensity Difference Between Adjacent Frames

Some background hotspot noise do not disappear completely but instead show a change in its intensity larger than the bleaching speed of the fluorescent dye. This type of noise cannot be removed by the aforementioned method. On the other hand, it is possible that a fluorescent signal that bleaches fast and disappears could be removed as noise. It is better to determine whether it is noise or not by the difference in intensity change of particles between frames.

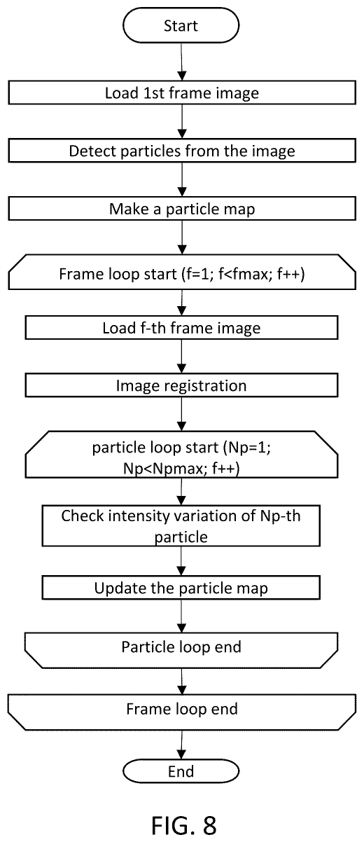

Another aspect of the background hotspot noise reduction method can be applied to the same system. In this case, the whole process follows the flowchart shown in FIG. 3, but the background hotspot noise reduction process is as shown in FIG. 8.

At first, the image of the first frame is loaded from the memory unit and processed for particle detection which detects bright spots in the image as particles, then the particle map is generated.

The next frame image is loaded and is applied image registration to align the current frame image with the first frame image.

Intensities of each detected particle based on the particle map are extracted from both the current frame image and from the previous frame image. In determining the intensity here, it is possible to use the maximum or average value for each detected particle. If the maximum value is used, the fluctuation will be large due to the influence of optical shot noise, and if the average value is used, the fluctuation will be small. Therefore, it is preferred to set the optimal intensity threshold value depending on the value to be used. The intensities difference is calculated and compared with an intensity threshold which is decided based on intensity of bleaching of the fluorescent dye. The intensity threshold should be set to an intensity change greater than the intensity change due to bleaching, so as not to exclude fluorescently labeled particles. In some embodiments, a single intensity threshold is set for a process. In others, the intensity threshold may be defined for each image (or for a set of images) based relative to an image baseline intensity or relative to a similar background intensity. When the intensity difference is larger than the intensity threshold, the particle is removed from the particle map. This intensity variation check is repeated for all detected particles in the particle map, then it is repeated for all frame images. Finally, the updated particle map is output.

Both processes for the background hotspot noise reduction can be applied simultaneously.

The temporal method is well-suitable for detecting the number of EVs, which means single channel detection. However, the acquisition and processing times can be longer than desired. Thus, processes to reduce the time needed for the analysis may be implemented.

### Exemplary Embodiments

In this embodiment, an image for particle detection and images for identifying background hotspot noise are separately acquired as shown in FIG. 7. First, the image acquisition parameter is set based on the wavelength used, the same as described above. Second, the image for particle detection is acquired with longer exposure time than that of the images for identifying background hotspot noise, which enables increased signal to noise ratio and to detect weak signals. Third, the images for identifying background hotspot noise is acquired as described above. Next, particle detection is applied to the image for particle detection and the particle map(s), which indicates particle position, are generated. Generating a background hotspot noise map process is applied which is similar to the aforementioned background reduction process shown in FIG. 4. This process generates a background hotspot noise map instead of the updated particle map by maintaining and updating the identified background hotspot noise positions. Finally, the process ends by excluding the identified background noise from the detected particle map, updating the particle map, and saving the various data. The background reduction process disclosed any of the embodiment described above are applied to this process of the background hotspot noise map.

The examples described herein were done using a Zeiss Axio Imager M2 with Zeiss Colibri-7 LED light source and Zeiss Plan Apochromat 40×/0.95 objective. Also, Chroma 39002, 39004, 39007 fluorescence filter sets were used for AF488, AF555, and AF647 channels. Either a Zeiss multiband filter or a Chroma fluorescence filter was used for Cy7 channel. The plasmonic chip(s) were set on the microscope stage and 50 images were acquired with 500 ms exposure time. In this experiment, images were acquired continuously for each channel.

### Effect

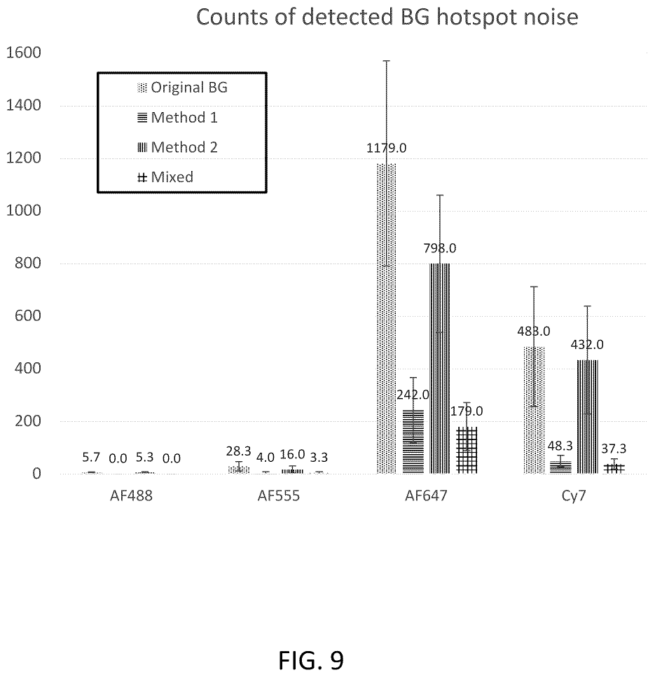

The effect of the background hotspot reduction methods disclosed in this invention is shown in FIG. 9. The background hotspot noise is analyzed using four different wavebands, which corresponded to the wavebands for AF488, AF555, AF647, and Cy7 fluorescent dyes. The original BG in FIG. 9 means the number of background hotspot noise in the first frame. The method 1 disclosed in the general method, the method 2 disclosed in the embodiment using particle detection result at each frame, and a mixed method of the method 1 and the method 2 were applied, and FIG. 9 shows the number of identified background hotspot noise. For these embodiments, the noise was rarely detected in AF488 and AF555 channels, there were many noises in AF647 and Cy7 channels. Thus, focusing on AF647 and Cy7 channels, and check the effect of disclosed methods. Overall, the method 1 is effective to reduce the noise, but the mixed method can reduce more noise which means the method 2 can reduce different property of noise. In method 2, mean value of detected particle area was used, and more than a 20% difference from the previous frame was identified as noise. Using the maximum value in the region or a lower intensity threshold value may detect noise more sensitively than Method 1. Method 2 does not have a lower limit, so it may not be able to detect noise that is below the detection level due to a gradual signal decrease. However, the fluorescent signal from the gently bleaching EV can remain. In AF647 channel, the method 1 can reduce about 79.5% of noise, whereas the mixed method can reduce 84.8% of noise in this experiment. Similarly, the method 1 can reduce about 90.1% of noise, and the mixed method can reduce 92.4% of noise in Cy7 channel.

The amount of noise reduction will depend on the exposure time and excitation power. For the embodiments as described above, it is preferable to set the acquisition time per frame to 600 ms or longer based on the detection limit of the desired signal. For a more stable signal, it is preferred to have an acquisition time per frame of 900 ms or longer. Considering the absolute number of hotspot noises detected, it is preferable to keep the acquisition time per frame below 2 s. The number of shots or images is determined by specifying the percentage of noise that remains without being identified as noise. When the above residual noise is set to percentages (e.g., 10%, 5%, and 2%) the number of acquired images can be changed based on this (e.g., 10 to 20 frames, 30 to 50 frames, and 70 to 90 frames, respectively.)

Absolute number of detected EVs is important for analysis of single EVs which can be from healthy cells and diseased cells such as cancer. When the plasmonic chip is used for single EVs analysis, the background hotspots noise is regarded as a signal from a single EV and it derives an incorrect analysis result without removal of the hotspots. Especially, in the case of low EV concentration, this error is significant since influence of the background is relatively larger than high EV concentration. Applying the background reduction method(s) of the preset disclosure, suppresses the error even in the case of low EV concentrations.

In some embodiments, to reduce the computational load, it is desirable to reduce the number of images taken; by considering the expected maximum noise duration time and the expected number of re-light of hotspot noise and using images taken at intervals of at least the product of the expected maximum noise duration time and the expected number of re-light of hotspot noise, the number of noise occurring at overlapping positions can be reduced, and effective reduction can be achieved with a smaller number of images. When multiple fluorescence channels are used, it is appropriate to take images of different channels one frame at a time and repeat this process to increase the interval between images taken of the same channel.

A different method for reducing computational cost in Method 1 is to not perform the particle detection analysis in each frame. By using the position information of particles in the base particle map and the location threshold value for the particle detection, the image intensity at each particle position in each frame is compared with the location threshold value, and if it is below the location threshold value, the particle is expected from the base particle map. This method can achieve the same results as the original method.

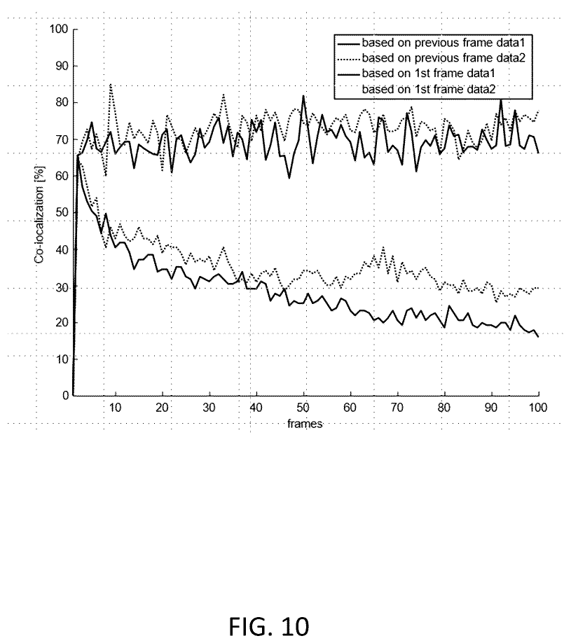

The co-localization rates comparing various embodiments are shown in FIG. 10. In this figure, data is shown for images in the AF647 channel, at 100 ms and 100 frames based on previous frame data and based on first frame data. The upper traces are based on previous frame data, showing around 70% overlap with the previous frame. The lower traces are based on first frame data, shown around 25% overlap with the first frame after 10 seconds.

### Definitions

In referring to the description, specific details are set forth in order to provide a thorough understanding of the examples disclosed. In other instances, well-known methods, procedures, components and circuits have not been described in detail as not to unnecessarily lengthen the present disclosure.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to be inclusive of end values and includes all sub-ranges subsumed therein, unless specifically stated otherwise. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g., substantially spherical, substantially perpendicular, substantially concentric, etc.) or a relative term (e.g., substantially similar, substantially the same, etc.).

Unless specifically stated otherwise, as apparent from the following disclosure, it is understood that, throughout the disclosure, discussions using terms such as “processing,” “computing,” “calculating,” “determining,” “displaying,” or the like, refer to the action and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order, unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

What is claimed is:

1. An optical imaging system for plasmon-enhanced optical imaging comprising:
a fluorescent microscope configured to image a sample on a plasmonic substrate;
an image acquisition control unit configured to acquire a first image at a first time and at least a second image(s) at a second time; and
an image processing unit configured to distinguish a signal within the at least a first image(s) as either (1) a signal from the sample or (2) a signal from a background hotspot, wherein distinguishing the signal comprises
executing a particle detection analysis on the first image and creating a base particle map,
executing a particle detection analysis on the at least a second image(s) and creating a temporal particle map,
defining a particle in the base particle map as a background hotspot when the particle has no correspond particle in the temporal particle map at a location within a location threshold of the particle, and
defining a particle in the base particle map as a signal from the sample when the particle has a correspond particle in the temporal particle map at a location within a location threshold of the particle.

2. The optical system of claim 1, wherein the execution of a particle detection analysis on the at least a second image(s) is performed only at the locations on the at least a second image(s) corresponding to the locations on the base particle map having detected particles.

3. The optical system of claim 1, wherein the background hotspot is removed from the base particle map to create an updated particle map.

4. The optical system of claim 3, wherein the updated particle map is further updated by the iterative process including:
executing a particle detection analysis on another image of the at least a second image(s) and creating a second (or later) temporal particle map,
defining a particle in the updated base particle map as a background hotspot when the particle has no correspond particle in the second (or later) temporal particle map at a location within a location threshold of the particle.

5. The optical system of claim 1, wherein the location threshold requires that more than half of the regions of the particles on the particle maps overlap.

6. The optical system of claim 1, wherein the particles detected in the base particle map and defined as a signal from the sample are extracellular vesicles (EVs) or EVs with one or more attached fluorescent dye(s).

7. The optical imaging system of claim 1, wherein the image acquisition control unit is configured to set an acquisition time for the first image and the second image, where the acquisition time is set to be less than a bleaching time of a florescent dye in the sample.

8. The optical imaging system of claim 1, wherein the acquisition time is between 100 ms and 5 s, and there are at least two images acquired at different times in the first channel.

9. The optical imaging system of claim 1, wherein distinguishing a signal further comprises:
registering the at least a second image with first image;
checking co-localization of the base particle map and the temporal particle map; and
updating the base particle map.

10. The optical imaging system of claim 1, wherein the image processing unit is further configured to:
subtract the signal from a background hotspot from the at least a second image(s);
combine the at least a second image(s) to form a combined image; and
send the combined image to a display.

11. The optical imaging system of claim 1, wherein the fluorescent microscope is a multi-channel fluorescent microscope and the image acquisition control unit is configured to acquire multiple images at different times in each of a first channel and a second channel, and wherein the image processing unit is configured to distinguish a signal within one or more of the multiple images in each of the first channel and the second channel.

12. The optical imaging system of claim 1, wherein the image acquisition control unit is further configured to acquire a high signal-to-noise ratio (SNR) image for data analysis, and wherein the at least the second image(s) acquired at different times are separately acquired for background identification.

13. The optical imaging system of claim 12, wherein the high SNR image is acquired before the at least a second image(s) for background identification, and wherein the acquisition time for the at least a second image(s) for background identification is between 10 ms and 1 s.

14. An analysis method comprising:
obtaining multiple images from a sample on a plasmonic substrate using a fluorescent microscope, the multiple images obtained at different times in a first channel; and
distinguishing a signal within one or more of the multiple images as either (1) a signal from the sample or (2) a signal from a background hotspot wherein distinguishing the signal comprises
executing a particle detection analysis on a first image of the multiple images and creating a base particle map,
executing a particle detection analysis on a second image of the multiple images and creating a temporal particle map,
defining a particle in the base particle map as a signal from the sample when the particle has a correspond particle in the temporal particle map at a location within a location threshold of the particle.

15. The analysis method of claim 14, further comprising:
setting an acquisition time for the multiple images that is less than a bleaching time of a florescent dye in the sample.

16. The analysis method of claim 14, wherein the acquisition time is between 100 ms and 900 ms, and there are at least two images acquired at different times in the first channel.

17. The analysis method of claim 14, wherein distinguishing a signal within one of the multiple images further comprises:
registering the second image from the multiple images with the base particle map;
checking co-localization of the base particle map and the temporal particle map; and
updating the base particle map.

18. The analysis method of claim 16, wherein the background hotspot is removed from the base particle map to create an updated particle map.

19. The analysis method of claim 18, further comprising displaying the updated base particle map.

20. The analysis method of claim 18, further comprising calculating the number of particles detected that are on the updated base particle map.

21. The analysis method of claim 16, further comprising
obtaining multiple images from each of at least two channels; and
distinguishing a signal within one or more of the multiple images from each of at least two channels.

22. The analysis method of claim 16, wherein the execution of a particle detection analysis the second image of the multiple images is performed only at the locations on the second image corresponding to the locations on the base particle map having detected particles.

23. The analysis method of claim 18, wherein the updated particle map is further updated by the iterative process including:
executing a particle detection analysis on another image of the multiple images and creating an additional temporal particle map,
defining a particle in the updated base particle map as a background hotspot when the particle has no correspond particle in the additional temporal particle map at a location within a location threshold of the particle.
